(19) United States
(12) Patent Application Publication
(10) Pub. No.: US 2014/0378916 A1
(43) Pub. Date: Dec. 25, 2014

(54) HYPOTUBE WITH ENHANCED STRENGTH AND DUCTILITY

(71) Applicant: Abbott Cardiovascular Systems, Inc., Santa Clara, CA (US)

(72) Inventor: John A. Simpson, Carlsbad, CA (US)

(21) Appl. No.: 13/925,559

(22) Filed: Jun. 24, 2013

(51) Int. Cl. A61L 31/02; A61L 31/14; A61M 25/00

(52) U.S. Cl. CPC A61L 31/022; A61M 25/0043; A61L 31/143; A61M 2025/0059
USPC 604/264

(57) ABSTRACT

Hypotubes for use with intra-corporal medical devices are fabricated from a stainless steel alloy exhibiting a combination of excellent yield strength with improved ductility as compared to cold worked AISI 304 stainless steel, from which hypotubes are typically fabricated. The stainless steel alloy may have: (1) a nitrogen content, a carbon content, or a combined nitrogen and carbon content that is greater than that allowed in AISI 304 stainless steel, providing an increased concentration of interstitial atoms to stabilize dislocations generated by cold work and/or (2) a combined nickel and manganese content that is lower than that allowed in AISI 304 stainless steel to reduce the stability of the austenitic structure, enabling a greater percentage of martensite to be stress-induced by a given level of cold work as compared to AISI 304 SS. Following cold working, the alloy may be heat treated to raise its yield strength by strain aging.

### BACKGROUND

Hypotubes are used extensively with various intra-corporal medical devices, such as rapid exchange balloon catheters, rapid exchange stent delivery catheters, and specialty guide wires including a hollow proximal shaft. For example, such hypotubes may be employed when introducing devices into intravascular sites within the body.

A hypotube needs appropriate strength and ductility to be able to resist kinking. For example, commercially available hypotubes are commonly formed of AISI 304 stainless steel, typically by seam welding flat strip material into tubing, and then drawing the welded tubing to a desired final size. Existing hypotubes are prone to kinking, particularly because their wall thickness is very thin relative to their diameter, as dictated by the size and geometry of the vasculature of the typical patient. Once a kink occurs, existing hypotubes can easily fracture, particularly if re-straightened, either deliberately or inadvertently.

One method for minimizing kinking of hypotubes is to simply increase the strength of the material by adding cold work. This may be achieved by drawing the tubing to a desired final size after welding, without annealing after cold drawing. When strengthening a hypotube by adding cold work, there is a natural tradeoff between increased strength and decreased ductility. Increasing the level of cold work increases the strength of the material, but simultaneously reduces its ductility (e.g., elongation to failure). Thus, while hypotubes that have been cold worked to increase strength may be less likely to begin kinking in the first place, once kinking begins to occur, the reduced ductility makes the hypotube more likely to fracture during the original kinking event or when subsequently re-straightened.

It would be advantageous to provide hypotubes that might exhibit great strength while maintaining relatively high ductility, effectively breaking the tradeoff between strength and ductility in stainless steel hypotubes.

### BRIEF SUMMARY

The present disclosure describes hypotubes for use with intra-corporal medical devices, such as rapid exchange balloon catheters, rapid exchange stent delivery catheters, specialty guide wires, and other devices introduced into the vasculature of a patient with the aid of a hypotube. Such hypotubes may include an elongate hollow body extending from a proximal end to a distal end, at least a portion of which is fabricated from a stainless steel alloy having: (1) a nitrogen content, a carbon content, or a combined nitrogen and carbon content that is greater than the upper specification limit for AISI 304 stainless steel; and/or (2) an austenitic stabilizer content (e.g., nickel and/or manganese) that is lower than the lower specification limit for AISI 304 stainless steel.

For example, AISI 304 stainless steels may include up to 0.08% carbon and up to 0.10% nitrogen, respectively, by weight. Increasing the fraction of these interstitial atoms surprisingly provides an alloy that can be static strain aged to exhibit strength characteristics (e.g., yield strength and/or ultimate tensile strength) that is approximately equal to or better than that provided by AISI 304 stainless steel, while at the same time providing ductility (e.g., elongation to failure) characteristics that are significantly higher than that provided by cold worked AISI 304 stainless steel. Such static strain aging can increase strength with little or no accompanying decrease in ductility.

In an embodiment, the combined nickel and manganese content of the stainless steel may be lower than that provided in an AISI 304 stainless steel. As nickel and manganese act as austenitic stabilizers, such an alloy will exhibit reduced austenitic (i.e., FCC structure) stability, thereby enabling a greater percentage of martensite to be stress-induced by a given level of cold work and thus providing a greater strain hardening rate. The manganese content of such stainless steels may be negligible (e.g., far less than its maximum specification limit of 2% by weight), so that it may be the nickel content which is lowered relative to the AISI 304 standard.

According to an embodiment the hypotube includes an elongate hollow body extending from a proximal end to a distal end, at least a portion of which is fabricated from a stainless steel alloy having a nickel content that is lower than that of AISI 304 stainless steel, and a nitrogen content that is greater than that of AISI 304 stainless steel. For example, it may be advantageous to limit the carbon content of such alloys (e.g., particularly those that are welded), as carbon within the heat affected zone surrounding the weld can result in chromium carbide precipitation at grain boundaries, which can result in reduced corrosion resistance within the heat affected zone of such welds. Thus, the nitrogen (rather than carbon) content may be increased relative to that provided within AISI 304 stainless steels to provide the desired interstitial atoms for stabilizing dislocations generated by prior cold work. In an embodiment, the carbon content may be minimized.

According to another embodiment the hypotube includes an elongate hollow body extending from a proximal end to a distal end, at least a portion of which is fabricated from a stainless steel alloy having a nickel content that is from 6% to 8% by weight, and a nitrogen content that is greater than 0.10% by weight. For example, AISI 304 stainless steel includes 8% to 10.5% nickel by weight, and no more than 0.10% nitrogen by weight. Because of its reduced nickel content, this embodiment exhibits reduced austenitic stability. Because of its increased nitrogen content, this embodiment includes a higher concentration of interstitial atoms that can stabilize dislocations generated by cold work. This embodiment may be strain aged (e.g., statically or dynamically strain aged) at a temperature (e.g., about 200° F. to about 400° F.) substantially below any annealing temperature that may be employed, which may aid in diffusing the interstitial atoms to stabilize such dislocations within the crystalline lattice structure, thereby boosting its yield strength.

These and other objects and features of the present disclosure will become more fully apparent from the following description and appended claims, or may be learned by the practice of the embodiments of the invention as set forth hereinafter.

### BRIEF DESCRIPTION OF THE DRAWINGS

To further clarify the above and other advantages and features of the present disclosure, a more particular description of the invention will be rendered by reference to specific embodiments thereof which are illustrated in the appended drawings. It is appreciated that these drawings depict only illustrated embodiments of the invention and are therefore not to be considered limiting of its scope. Embodiments of the invention will be described and explained with additional specificity and detail through the use of the accompanying drawings in which:

FIG. 1 illustrates a partial cut-away view of an exemplary guide wire device including a hypotube proximal portion according to one embodiment of the present disclosure;

FIG. 2A is a side elevation view of an exemplary rapid exchange stent delivery catheter including a hypotube proximal portion;

FIG. 2B is a side elevation view, in cross-section, of the stent delivery catheter of FIG. 2A the distal end of the catheter being disposed within a body lumen for placement of a stent;

FIG. 3 is an elevational view, partially in section, showing the radiopaque stent of FIG. 2 expanded within the lumen after withdrawal of the delivery catheter;

FIG. 4 shows stress-strain curve data for a stainless steel alloy sheet including lower nickel content and higher nitrogen content than the specification limits of AISI 304 stainless steel, processed with various levels of cold work and static strain aging;

FIG. 5A shows ductility versus yield strength and ultimate tensile strength data for AISI 304 stainless steel sheet;

FIG. 5B shows ductility versus yield strength and ultimate tensile strength data for AISI 301LN stainless steel sheet;

FIG. 6A compares ductility versus ultimate tensile strength for AISI 304 and AISI 301LN stainless steels at various levels of cold work;

FIG. 6B compares ductility versus yield strength for AISI 304 and AISI 301LN stainless steels at various levels of cold work;

FIG. 7A shows ductility versus ultimate tensile strength for AISI 304 stainless steel sheet, AISI 301LN stainless steel sheet, as well as ductility versus ultimate tensile strength for two differently sized hypotubes fabricated from conventional AISI 304 stainless steel; and

FIG. 7B shows ductility versus yield strength for AISI 304 stainless steel sheet, AISI 301LN stainless steel sheet, as well as ductility versus yield strength for two differently sized hypotubes fabricated from conventional AISI 304 stainless steel.

### DETAILED DESCRIPTION

### I. Introduction

In one aspect, the present disclosure describes hypotubes exhibiting increased strength and ductility as compared to stainless steel hypotubes fabricated from AISI 304 stainless steel, the material typically employed in such fabrication of hypotubes. Such hypotubes may be employed in or with a wide variety of intra-corporal medical devices. Examples of such devices include, but are not limited to, rapid exchange balloon catheters, rapid exchange stent delivery catheters, and guide wires including a hollow hypotube shaft portion. The hypotube may be fabricated from a stainless steel alloy having a nitrogen content, a carbon content, or a combined nitrogen and carbon content that is greater than that of AISI 304 stainless steel. Such increased interstitial atom concentration provides not only an increased rate of work hardening during cold work, but also enables stabilization of cold work generated dislocations within the lattice structure of the alloy by virtue of a “strain aging” heat treatment. The alloy may purposely include a reduced concentration of austenitic stabilizing elements (i.e., nickel and manganese) as compared to AISI 304 stainless steel, reducing the stability of the austenitic structure so that a greater percentage of martensite is stress induced by a given level of cold work, thus further increasing its rate of work hardening. Such a characteristic enables a given level of strength to be attained with less associated reduction in ductility as compared to AISI 304 stainless steel.

### II. Exemplary Hypotubes

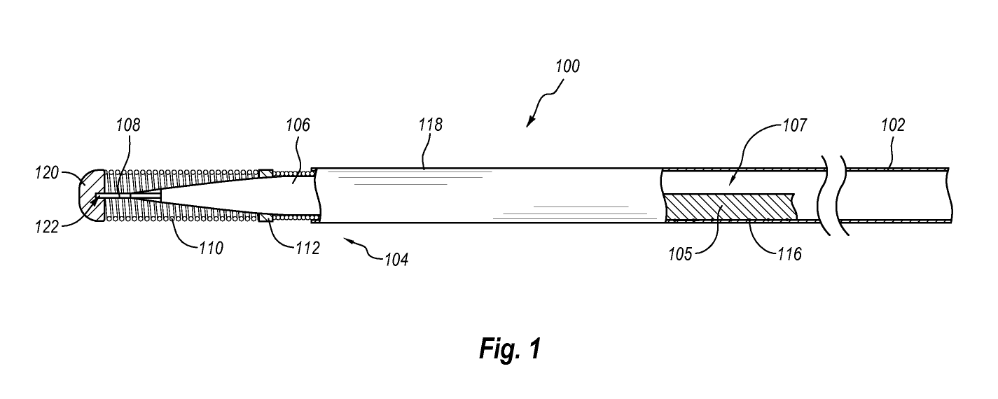

Referring now to FIG. 1, a partial cut-away view of an example of a guide wire device 100 that embodies features of the invention is illustrated. The guide wire device 100 may be adapted to be inserted into a patient's body lumen, such as an artery or another blood vessel. The guide wire device 100 includes an elongated hollow hypotube proximal portion 102 and a distal portion 104. Although not limited to such, the hypotube portion 102 may enable hydraulic, electrical, and/or optical communication between its distal and proximal ends. A core wire portion 105 of guide wire device 100 may be joined to hypotube portion 102 via a welded or other joint 116. Core wire portion 105 may be sufficiently small in diameter to allow clearance 107 for hydraulic pressure communication, passage of an electrical or optical fiber, etc. between core wire 105 and hypotube 102. In one embodiment, at least the elongated hollow hypotube proximal portion 102 may be formed from a stainless steel alloy as described herein providing increased kink-related damage tolerance as compared to AISI 304 stainless steel. Distal portion 104, including core wire 105, may be formed of a similar or different material (e.g., a Ni—Ti alloy) as compared to proximal portion 102. In embodiments where the elongated proximal portion 102 and the distal portion 104 or core wire 105 are formed from different materials, the elongated hollow hypotube proximal portion 102 and the distal portion 104, including core wire 105, may be coupled to one another via a welded or other joint 116 that couples the hollow hypotube proximal portion 102 and the distal portion 104 into a torque transmitting relationship.

Distal portion 104 may have at least one tapered section 106 that, in the illustrated embodiment, becomes smaller in the distal direction. The length and diameter of the tapered distal core section 106 can, for example, affect the trackability of the guide wire device 100. Typically, gradual or long tapers produce a guide wire device with less support but greater trackability, while abrupt or short tapers produce a guide wire device that provides greater support but also greater risk of prolapse (i.e., kinking) when steering. The length of the distal end section 106 can, for example, affect the steerability of the guide-wire device 100. In one embodiment, the distal end section 106 is about 10 cm to about 40 cm in length. In another embodiment, the distal end section 106 is about 2 to about 6 cm in length, or about 2 to 4 cm in length. Tapered distal core section 106 may further include a shapeable distal end section 108.

Guide wire device 100 may include a helical coil section 110. The helical coil section 110 affects support, trackability, and visibility of the guide wire device and provides tactile feedback. In some embodiments, the most distal section of the helical coil section 110 is made of radiopaque metal, such as platinum or a platinum-nickel or platinum-iridium alloy, to facilitate the observation thereof while it is disposed within a patient's body. As illustrated, the helical coil section 110 may be disposed about at least a portion of the distal portion 104 and may have a rounded, atraumatic cap section 120 on the distal end thereof. The helical coil section 110 may be secured to the distal portion 104 at proximal location 114 and at intermediate location 112 by a suitable technique such as, but not limited to, soldering, brazing, or welding. Distal end section 108 may similarly be secured to the rounded, atraumatic cap section 120 by virtue of a joint 122 such as, but not limited to, a soldered, brazed, or welded joint.

In one embodiment, portions of the guide wire device 100 are coated with a coating 118 of lubricous material such as polytetrafluoroethylene (PTFE) (sold under the trademark Teflon by du Pont, de Nemours & Co.) or other suitable lubricous coatings such as polysiloxane (silicone) coatings, polyvinylpyrrolidone (PVP), and the like.

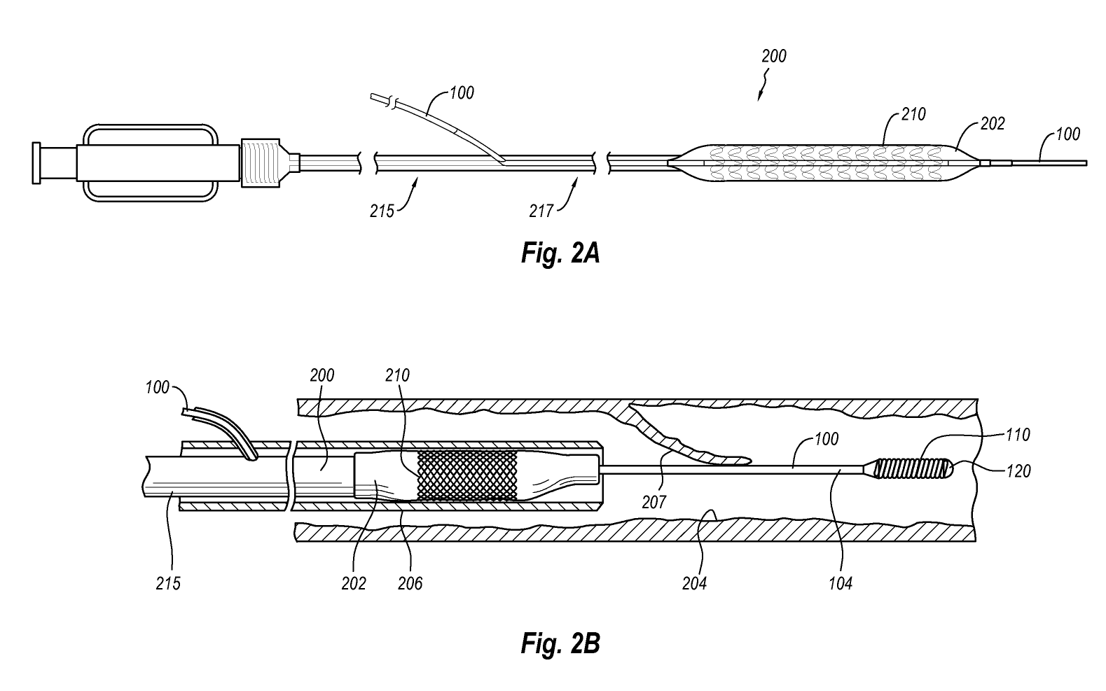

FIG. 2A shows an exemplary rapid exchange stent delivery catheter 200, which may employ a hypotube fabricated according to the present disclosure. Catheter 200 may include a proximal hypotube portion 215 that may be fabricated from a stainless steel other than AISI 304 as described herein. The distal portion 217 of catheter 200 may be formed of a suitable polymer material (e.g., including a polymer outer member and polymer inner member). Guide wire 100 is inserted through the inner member. FIG. 2B shows the rapid exchange stent delivery catheter being used to deploy a stent 210 within the vasculature of a patient. A guide wire device 100 is shown configured to facilitate deploying a stent 210. The portion of the illustrated guide wire device 100 that can be seen in FIG. 2B includes a distal portion 104, a helical coil section 110, and an atraumatic cap section 120. The delivery catheter 200 may have an expandable member or balloon 202 for expanding the stent 210, on which the stent 210 is mounted, within a body lumen 204 such as an artery. In another embodiment, stent 210 may be self-expanding. For example, a sheath may be initially disposed over stent 210 so as to maintain an un-expanded configuration. When stent 210 is advanced to a desired position, the sheath may be removed and the stent 210 expanded.

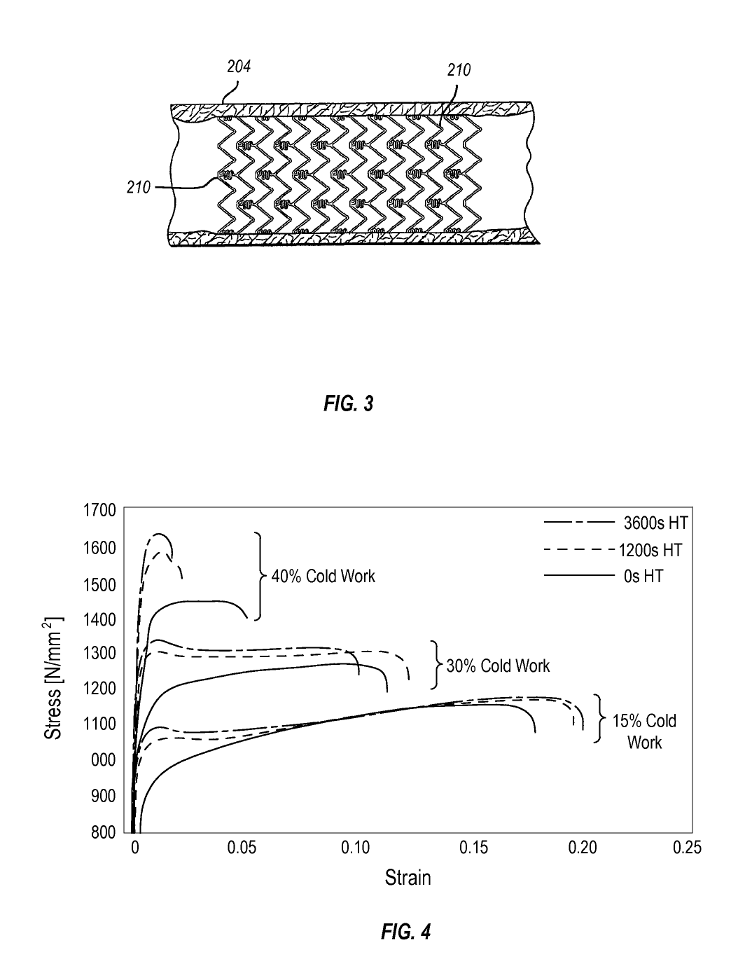

Referring to FIG. 2B, in use, the stent 210 may be mounted onto the inflatable balloon 202 on the distal extremity of the delivery catheter 200. The balloon 202 may be slightly inflated to secure the stent 210 onto an exterior of the balloon 202. The catheter/stent assembly may be introduced within a living subject using a conventional Seldinger technique through a guiding catheter 206. The guide wire 100 may be disposed across the damaged arterial section with the detached or dissected lining 207 and then the catheter/stent assembly 200/210 may be advanced over the guide wire 100 within the body lumen 204 until the stent 210 is directly under the detached lining 207. The balloon 202 of the catheter 200 may be expanded, expanding the stent 210 against the interior surface defining the body lumen 204 by, for example, permanent plastic deformation of the stent 210. In an embodiment employing a self-expanding stent, removal of a sheath may be sufficient to allow a self-expanding stent to expand against the interior surface defining body lumen 204. In either case, when deployed, the stent 210 holds open the body lumen 204 after the catheter 200 and the balloon 202 are withdrawn. FIG. 3 shows the implanted stent 210 positioned in the vessel 204 after the balloon 202 has been deflated and the catheter 200 and guide wire 100 have been withdrawn from the patient.

The delivery catheter 200 may be similar to conventional balloon dilatation catheters commonly used for angioplasty procedures, but including a hypotube 215 at a proximal end of catheter 200 that may be fabricated according to the present disclosure. The balloon 202 may be formed of, for example, polyethylene, polyethylene terephthalate, polyvinylchloride, nylon, Pebax™ or another suitable polymeric material, which may be attached to the polymer outer member of catheter 200. To facilitate the stent 210 remaining in place on the balloon 202 during delivery to the site of the damage within the body lumen 204, the stent 210 may be compressed onto the balloon 202. Other techniques for securing the stent 210 onto the balloon 202 may also be used, such as providing collars or ridges on edges of a working portion (i.e., a cylindrical portion) of the balloon 202.

As the hypotube portion 215 of catheter 200 may often need to be advanced through tortuous vasculature, with conventionally employed AISI 304 stainless steel, there may be a tendency for the hypotube to kink while attempting to navigate a bend. As described above, such a kink can result in fracture of the relatively thin-walled hypotube, particularly if the kink is restraightened, either deliberately or inadvertently. The hypotube is: (1) formed of a stainless steel alloy including a higher nitrogen content, carbon content, or combined nitrogen content as compared to the upper specification limit of AISI 304 stainless steel; and/or (2) formed of a stainless steel alloy including a lower concentration of austenitic stabilizers (e.g., nickel and/or manganese) as compared to the lower specification limit of AISI 304 stainless steel. As a result of (1), the stainless steel alloy can be strain aged to better stabilize dislocations within the lattice structure resulting from cold work. While conventional wisdom may hold that cold working the stainless steel alloy is the only hardening mechanism (with its inherent tradeoff of reduced ductility for increased yield strength), the inventor has found that a hypotube can exhibit a substantial gain in yield strength as a result of heat treating the alloy including an elevated concentration of interstitial atoms, while exhibiting negligible or no reduction in ductility.

Because the hypotube portions of such medical devices are formed of a stainless steel alloy including: (1) a higher nitrogen and/or carbon content as compared to the upper specification limit of AISI 304 stainless steel; and/or (2) a lower austenitic stabilizer (e.g., nickel and manganese) content as compared to the lower specification limit of AISI 304 stainless steel, such hypotube portions can exhibit increased kink-related damage tolerance (i.e., a reduced tendency to kink in the first place, as well as a reduced tendency to break during an original kinking event or during subsequent re-straightening, whether deliberate or inadvertent).

Reducing the concentration of any austenitic stabilizers, such as nickel and manganese, within the stainless steel alloy reduces the stability of the austenitic (FCC) structure, enabling a greater percentage of martensite (i.e., “alpha-prime” martensite) to be stress-induced by a given level of cold work. As a result, less cold working of the stainless steel alloy is required to achieve a desired level of strength. As cold work which increases strength simultaneously results in decreased ductility (e.g., as measured by percent elongation to failure), the tradeoff between strength and ductility is less severe where martensite formation is more readily stress-induced. In other words, for a stainless steel alloy with lower combined nickel and manganese content, a given level of yield and/or ultimate tensile strength achieved through cold working of the allow may simultaneously provide a higher level of ductility as compared to a stainless steel alloy that is similar, but with higher combined nickel and manganese content. This result is possible because less cold work is required to achieve the given level of strength in the stainless steel alloy including a lower level of combined nickel and manganese.

Providing a lower level of austenitic stabilizers can also provide a hypotube fabricated therefrom with the ability to spontaneously exhibit localized hardening when a kinking event begins to occur. For example, because the alloy exhibits lower austenitic stabilization, where a kink begins to form, the associated permanent deformation causes the alloy to locally harden to a greater degree than AISI 304 stainless steel. This superior localized spontaneous kink-induced hardening provides greater resistance to further deformation under the loading conditions which initiated kinking. In other words, such an alloy will better resist further progression of an existing kink than an AISI 304 stainless steel hypotube of equivalent dimensions and initial strength characteristics.

The stainless steel alloy may include a higher interstitial atom content than that provided by AISI 304 stainless steel. Such interstitial atoms are typically nitrogen and/or carbon atoms. Because of the higher interstitial atom content, such atoms are available to diffuse to and stabilize dislocations within the crystal lattice structure, which dislocations may be generated by cold work. For example, the stainless steel alloy may be heat treated at a relatively low temperature, below any annealing temperature, causing the interstitial atoms to diffuse to and thus stabilize such dislocations. Thus, by purposely maintaining a nitrogen, carbon, or combined nitrogen and carbon concentration at a relatively high level, a substantial strain aging response can be achieved by such heat treatment. Such static strain aging can increase the strength of the alloy, without any significant associated decrease in ductility. Such a characteristic is very advantageous, as it allows the fabricated hypotube to exhibit both high strength and high ductility—much higher ductility than that possible with ordinarily employed AISI 304 stainless steel used in commercial hypotubing. Such advantages are readily apparent in the data presented in FIGS. 4-7B, discussed in further detail below.

Temperatures associated with such a static strain aging treatment may be from about 150° F. to about 750° F., from about 200° F. to about 500° F., or from about 200° F. to about 400° F. In another embodiment, the temperature may range from about 160° C. to about 400° C. Exposure times associated with such a static strain aging treatment may be depend on the particular temperature selected (e.g., with lower temperatures, higher exposure times may be desired, and may typically range from about 5 minutes to about 10 hours, from about 10 minutes to about 2 hours, or from about 20 minutes to about 1 hour.

Because high carbon content can result in decreased corrosion resistance in a heat affected zone (i.e., weld sensitization as a result of chromium carbide precipitation at grain boundaries), in an embodiment the carbon content may be maintained at a relatively low level (i.e., minimized). As such, in an embodiment, nitrogen may be employed as the principal interstitial atom for providing a substantial static strain aging response. In other words, the nitrogen content of the stainless steel alloy may be higher than the upper specification limit allowed by AISI 304 stainless steel. The carbon content may be no greater than the maximum allowed in AISI 304 stainless steels. In an embodiment, the carbon content may be maintained at a particularly low level (e.g., corresponding to an AISI 304L carbon level), such as not greater than 0.03% by weight.

Where the stainless steel alloy employed includes significant carbon content, the initial as-welded tubing may be solution-annealed prior to drawing into hypotubing in order to re-dissolve chromium carbide that may have precipitated at grain boundaries within the heat affected zone adjacent the weld. Where the stainless steel alloy employed includes very low or negligible carbon content (e.g., no more than 0.03% carbon by weight), no post weld solution-annealing step may be needed, as little to no chromium carbide may precipitate as a result of the weld. Where the stainless steel alloy is solution-annealed after welding, the temperature may be at least about 1000° C., from about 1000° C. to about 1500° C., or from about 1000° C. to about 1200° C. The static strain aging heat treatment described above may be achieved at a temperature well below the solution-annealing temperature.

By way of non-limiting example, various AISI 300 series stainless steels exhibiting one or more of the above characteristics may be employed. For example, AISI 304N, AISI 304LN, AISI 301, AISI 301L, AISI 301LN, AISI 316N, AISI 316LN, and AISI 302 stainless steels may be employed, as they include lower concentrations of austenitic stabilizing elements (e.g., nickel and manganese) and/or higher concentrations of nitrogen and/or carbon. Other stainless steels exhibiting such characteristics may also be suitable for use.

Nitrogen enriched stainless steels (e.g., those designated “N”) may be particularly beneficial where an elevated nitrogen content is desired for attaining a substantial static strain aging response through heat treatment as described above. Nitrogen enriched stainless steels with low carbon content (e.g., at least those designated “LN”) may be particularly beneficial, as they include both elevated nitrogen content, which provides the desired static strain aging response, as well as low carbon content so that the alloys can be welded with minimal reduction in corrosion resistance (a.k.a. “sensitization) within the heat affected zone due to chromium carbide formation.

According to an embodiment, the stainless steel alloy may have a nickel content that is at or below 8% by weight (e.g., 6% to 8% by weight). As seen in Table 1 below, any of the AISI 301 series stainless steels (e.g., 301, 301L, 301LN) are examples of stainless steel alloys that include less than 8% nickel by weight. Several of the other AISI 300 series stainless steels (e.g., 304N, 304LN, and 302) can include 8% nickel by weight at the lower end point of the standard.

The stainless steel alloy may have a nitrogen content that is greater than 0.10% by weight (e.g., greater than 0.10% to about 0.30% by weight, or greater than 0.10% to about 0.20% by weight). Examples of such AISI stainless steels that may include more than 0.10% nitrogen by weight include 304N, 304LN, 301L, 301LN, 316N, and 316LN. The stainless steel alloy may have a combined nitrogen and carbon content that is greater than 0.18% by weight (e.g., 0.18% is the maximum combined nitrogen and carbon allowed by the AISI 304 standard). For example, as seen in Table 1 below, each of AISI stainless steels 304N, 304LN, 301L, 301LN, 316N, and 316LN may include a combined nitrogen and carbon content greater than 0.18% by weight.

An embodiment may have a carbon content that is greater than 0.08% by weight (e.g., greater than 0.08% to about 0.15% by weight). For example, AISI 301 and 302 may have carbon contents greater than 0.08% carbon by weight. In another embodiment, the stainless steel alloy may have a carbon content that is not greater than 0.03% by weight. AISI 304LN, 301L, 301LN, and 316LN are examples of such stainless steel alloys which have carbon contents no greater than 0.03% by weight.

TABLE 1

| Stainless | Cr | Ni | Mn | Si | N | P | C | S | Mo | Fe |
|---|---|---|---|---|---|---|---|---|---|---|
| Steel | (wt %) | (wt %) | (wt %) | (wt %) | (wt %) | (wt %) | (wt %) | (wt %) | (wt %) | (wt %) |
| 304 | 18-20 | 8-10.5 | 2 max | 0.75 | 0.10 max | 0.045 | 0.08 | 0.03 max | — | Balance |
| | | | | max | | max | max | | | |
| 304N | 18-20 | 8-10.5 | 2 max | 1 max | 0.10-0.16 | 0.045 | 0.08 | 0.03 max | — | Balance |
| | | | | | | max | max | | | |
| 304LN | 18-20 | 8-12 | 2 max | 1 max | 0.10-0.16 | 0.045 | 0.03 | 0.03 max | — | Balance |
| | | | | | | max | max | | | |
| 304LN* | 18-20 | 8-10.5 | 2 max | 1 max | 0.10-0.15 | 0.045 | 0.03 | 0.03 max | — | Balance |
| | | | | | | max | max | | | |
| 301 | 16-18 | 6.0-8.0 | 2 max | 1 max | 0.1 max | 0.045 | 0.15 | 0.03 max | — | Balance |
| | | | | | | max | max | | | |
| 301L | 16-18 | 6.0-8.0 | 2 max | 1 max | 0.20 max | 0.045 | 0.03 | 0.03 max | — | Balance |
| | | | | | | max | max | | | |
| 301LN | 16-18 | 6.0-8.0 | 2 max | 1 max | 0.07-0.20 | 0.045 | 0.03 | 0.03 max | — | Balance |
| | | | | | | max | max | | | |
| 302 | 17-19 | 8-10 | 2 max | 1 max | — | 0.045 | 0.15 | 0.03 max | — | Balance |
| | | | | | | max | max | | | |
| 316N | 16.5-18.5 | 11-14 | 2 max | 1 max | 0.12-0.22 | 0.045 | 0.08 | 0.03 max | 2.5-3.0 | Balance |
| | | | | | | max | max | | | |
| 316N* | 16-18 | 10-14 | 2 max | 1 max | 0.10-0.16 | 0.045 | 0.08 | 0.03 max | 2.0-3.0 | Balance |
| | | | | | | max | max | | | |
| 316LN | 16.5-18.5 | 11-14 | 2 max | 1 max | 0.12-0.22 | 0.045 | 0.03 | 0.03 max | 2.5-3.0 | Balance |
| | | | | | | max | max | | | |
| 316LN* | 16-18 | 10-14 | 2 max | 1 max | 0.10-0.30 | 0.045 | 0.03 | 0.03 max | 2.0-3.0 | Balance |
| | | | | | | max | max | | | |

*Different sources list slightly different standards for at least some of the AISI stainless steels.

AISI 316N and 316LN are examples of AISI stainless steels including molybdenum. The stainless steels listed in Table 1 typically include no or negligible manganese (e.g., less than 2% manganese by weight). AISI 301, 301L, and 301LN are examples of stainless steels that include not more than 8% nickel by weight. Any of the “N” designated stainless steels, as well as AISI 301L are examples of stainless steels that may include more than 0.10% nitrogen by weight. The use of AISI 301LN may be particularly beneficial, as the standard requires a more narrowly defined range of nitrogen content as compared to 301L (i.e., 0.07%-0.20% as compared to 0.20% maximum). Such a more controlled nitrogen content may exhibit greater lot-to-lot repeatability in work and static strain aging hardening for quality control manufacturing purposes. Both 301L and 301LN also exhibit very low carbon levels (i.e., no more than 0.03%) so that such alloys may not require any post weld annealing treatment to re-dissolve chromium carbide within the heat affected zone.

The stainless steel may include small amounts (e.g., less than 2%, less than 1%, or less than 0.5% by weight) of other trace elements, such as, but not limited to copper, cobalt, and tin. Table 2 shows another example of a stainless steel material that may also be suitable for use in hypotube fabrication according to the present disclosure.

TABLE 2

| Cr | Ni | Mn | Si | N | P | C | Mo | Cu | Co | Sn | Fe |
|---|---|---|---|---|---|---|---|---|---|---|---|
| (wt %) | (wt %) | (wt %) | (wt %) | (wt %) | (wt %) | (wt %) | (wt %) | (wt %) | (wt %) | (wt %) | (wt %) |
| 17.50 | 6.50 | 1.260 | 0.450 | 0.123 | 0.030 | 0.022 | 0.21 | 0.220 | 0.100 | 0.005 | Balance |

The alloy of table 2 is similar to an AISI 301L or 301LN alloy, but includes small amounts of molybdenum, copper, cobalt, and tin. The stainless steel alloy of Table 2 exhibits stress-strain curves as shown in FIG. 4 for sheet material cold rolled to 15%, 30%, and 40% reductions (see T. Juuti et al, Static Strain Aging in Some Austenitic Stainless Steels, Materials Science Forum Vols. 638-642, Thermec 2009, pp. 3278-3283). Data is shown for the alloy of Table 2 that has not been heat treated, that has been heat treated (at 190° C.) for a period of 20 minutes, and that has been heat treated for a longer period of 60 minutes. The results indicate that a static strain aging response can be achieved with such a material. Advantageously, such a hardening response can be achieved with no or minimal decrease in ductility.

According to an embodiment, medical devices including a hypotube portion fabricated from a stainless steel alloy as described herein may be manufactured by providing the stainless steel alloy in flat strip form, which is subsequently bent edge to edge to form a tubular structure. The edges may be seam welded or otherwise joined together, resulting in the desired tube shape. Once welded, the tube may be drawn (e.g., cold drawn) to a final desired size. Cold drawing can increase yield strength as described above. After drawing (whether cold drawn or otherwise), the stainless steel alloy tubing may be heat treated at a sub-annealing temperature as described above to produce a strain aging response, providing increased yield strength, which may be associated with no or negligible decrease in ductility. Strain aging may be achieved statically (i.e., without simultaneous deformation) or dynamically (i.e., as deformation is being applied).

In order to minimize any tendency for a heat affected zone of the hypotube adjacent the seam weld to corrode, the carbon content of the stainless steel alloy may be minimized, preferentially employing nitrogen rather than carbon as the interstitial atom used in stabilizing dislocations. For example, the stainless steel alloy may exhibit a carbon content not greater than 0.03% by weight. Alternatively, where the carbon content is greater, the tubing may be annealed post welding, in order to re-dissolve any precipitated chromium carbide.

The amount of cold work may be lower than that typically provided when forming a hypotube from AISI 304 stainless steel. For example, in an embodiment, the cold-worked section(s) may include about 5% to about 50% cold work, about 5% to about 40% cold work, about 10% cold work to about 40% cold work, or about 15% to about 30% cold work. As described above, where the concentration of austenitic stabilizers in the stainless steel alloy (e.g., nickel and/or manganese) is lower than that allowed within AISI 304 stainless steel, a greater percentage of martensite can be stress-induced by a given level of cold work, more quickly providing a desired level of yield strength (and preserving greater ductility as a result of the lower cold work). As described above, the stainless steel alloy may subsequently be heat treated at a relatively low (i.e., sub annealing) temperature to further increase yield strength without sacrificing any significant degree of ductility through diffusion of interstitial atoms (e.g., nitrogen and/or carbon) to stabilize cold work induced dislocations.

While the compositional differences between the stainless steel alloys employed according to the present disclosure may appear similar to AISI 304 stainless steel, the differences of decreased austenitic stabilizer content and/or increased interstitial atom content can provide surprising benefits—namely, the ability to break the tradeoff between strength and ductility. In other words, such differences provide a stainless steel alloy which surprisingly does not force one to choose between increased strength versus maintaining a desired relatively high level of ductility. Rather, such a stainless steel alloy exhibiting slightly different compositional characteristics as compared to AISI 304 stainless steel provides markedly superior properties in terms of providing both high yield strength and high ductility.

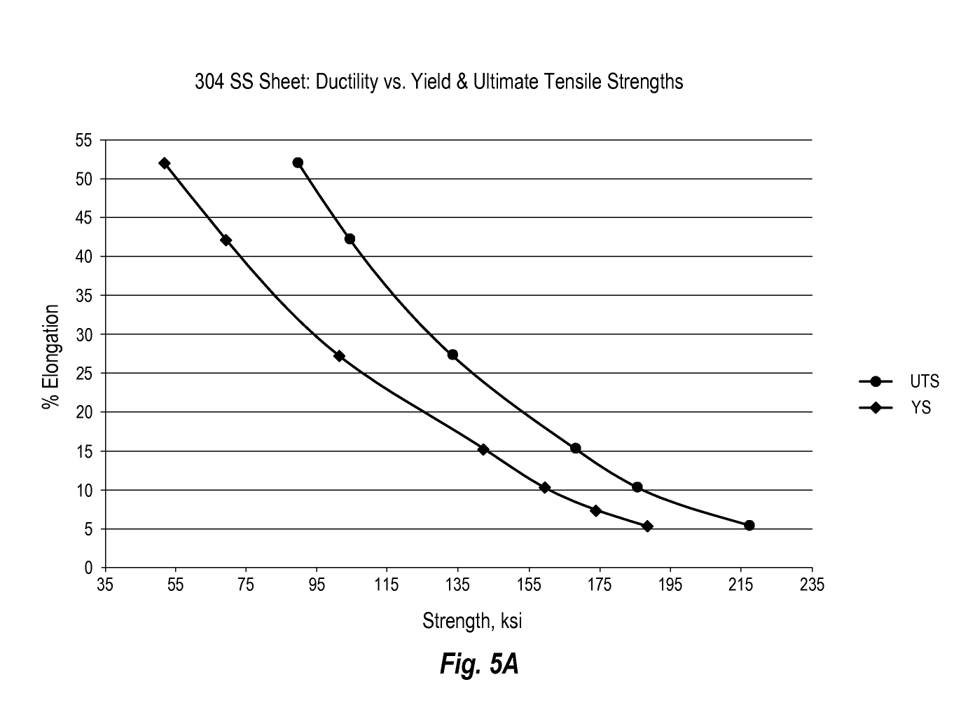

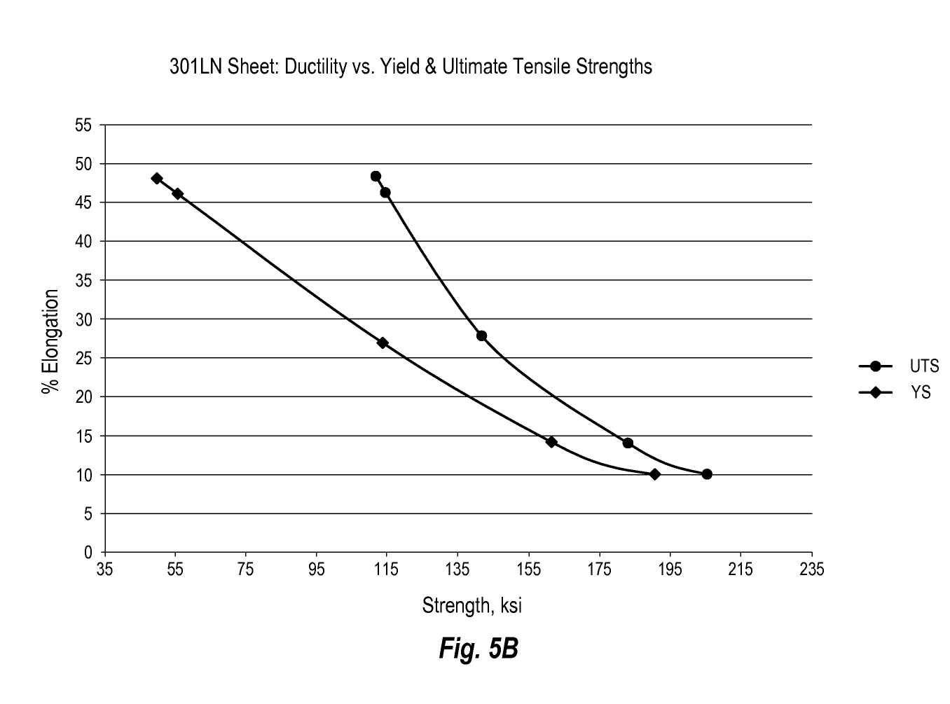

FIGS. 5A-7B quantitatively illustrate these advantages. FIG. 5A shows ductility versus ultimate tensile strength (“UTS”) for AISI 304 stainless steel sheet material. The data in FIG. 5A was taken from technical datasheets from a European supplier of AISI 304 stainless steel sheet (Uginox 18-9E, 18-9D, and 18-9DDQ). At higher UTS values, the material provides less ductility. For example, where a UTS from 175 ksi to 215 ksi (e.g., 195 ksi) is desired, AISI 304 stainless steel only provides a ductility value of well below 10% (e.g., perhaps even lower than 5%). FIG. 5B shows both ductility versus UTS values and ductility versus yield strength (YS) for AISI 301LN sheet material. The data in FIG. 5B was taken from technical datasheets from a European supplier of AISI 301LN stainless steel sheet (Uginox 18-7L). For example, at a UTS of 195 ksi, the AISI 301LN stainless steel alloy provides over 10% elongation (e.g., about 12-13%). At UTS values above 200 ksi, the elongation to failure characteristics of AISI 301LN can be twice that provided by AISI 304 stainless steel. Such a difference may be beneficial in preventing a kink from occurring in the first place, and would help in minimizing any tendency of such a kink to result in fracture of a hypotube upon subsequent re-straightening.

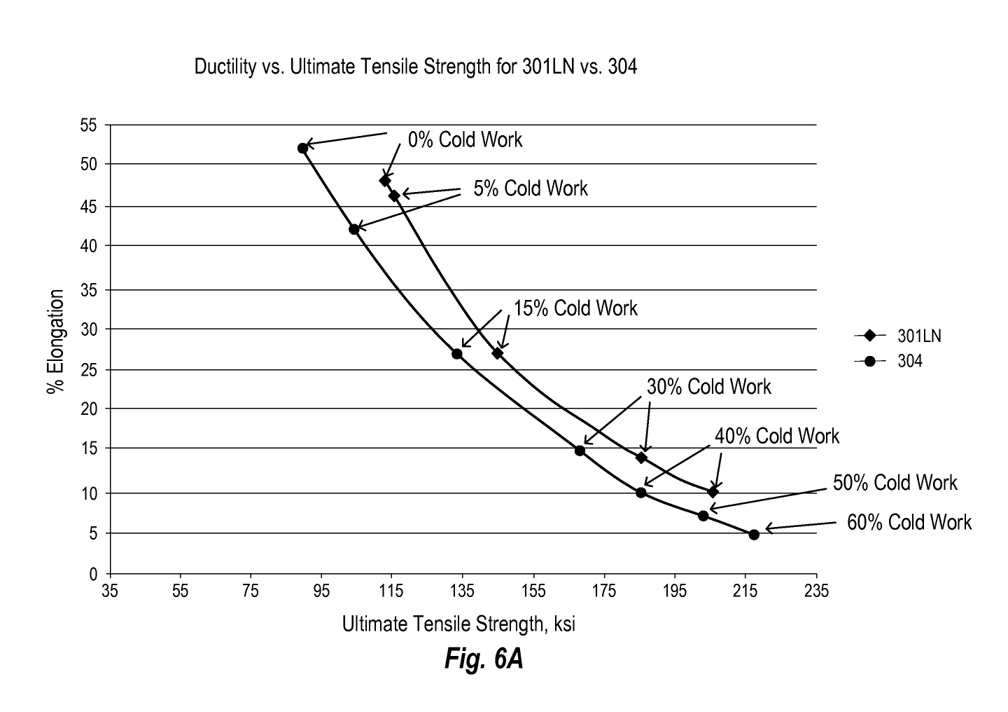

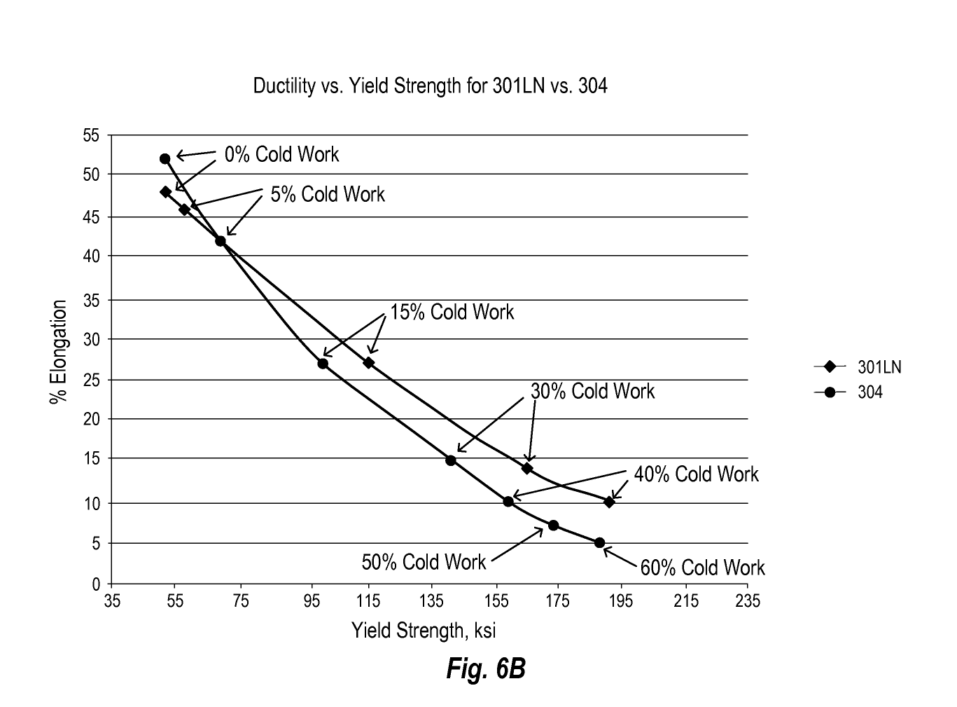

FIG. 6A shows ductility versus UTS curves for both AISI 304 stainless steel and AISI 301LN stainless steel, where the increase in UTS is provided by cold work. The data in FIG. 6A was taken from technical datasheets from a European supplier of AISI 301LN and 304 stainless steel sheet (Uginox 18-7L vs. Uginox 18-9E, 18-9D, and 18-9DDQ). By way of example, in order to provide a UTS of 195 ksi in AISI 304 stainless steel, the alloy must be cold worked to nearly 50%, and will exhibit ductility of less than 10% elongation to failure (e.g., about 8%). By comparison, the AISI 301LN stainless steel only needs to be cold worked to about 35% to exhibit the same UTS, and will exhibit ductility of over 10% elongation to failure (e.g., about 12-13%). FIG. 6B shows similar data as presented in FIG. 6A, but for yield strength rather than UTS. In FIG. 6B, the same trend is observed as seen in FIG. 6A—that the yield strength of AISI 301LN provides significantly greater ductility than AISI 304 when cold worked to achieve high yield strength (e.g., 160-190 ksi). The data in FIG. 6B was taken from technical datasheets from a European supplier of AISI 301LN and 304 stainless steel sheet (Uginox 18-7L vs. Uginox 18-9E, 18-9D, and 18-9DDQ).

It is noted that a desired level of yield strength or ultimate tensile strength does not have to be fully provided through cold working of the stainless steel alloy, but that at least a portion of the hardening can be achieved through a low temperature static strain aging treatment, preserving even greater ductility.

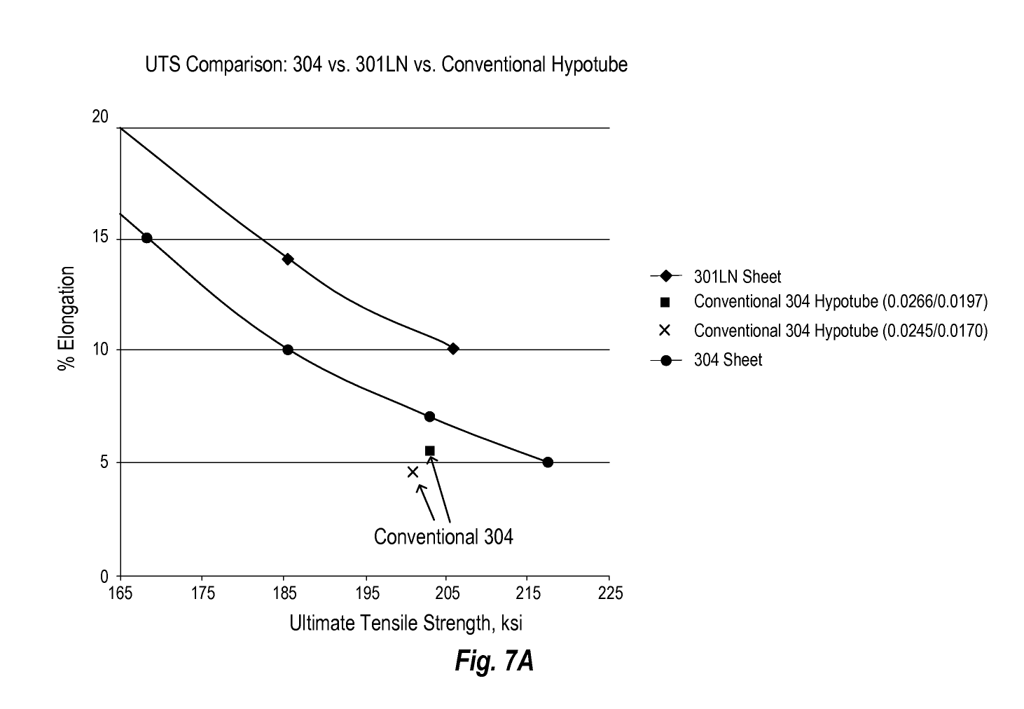

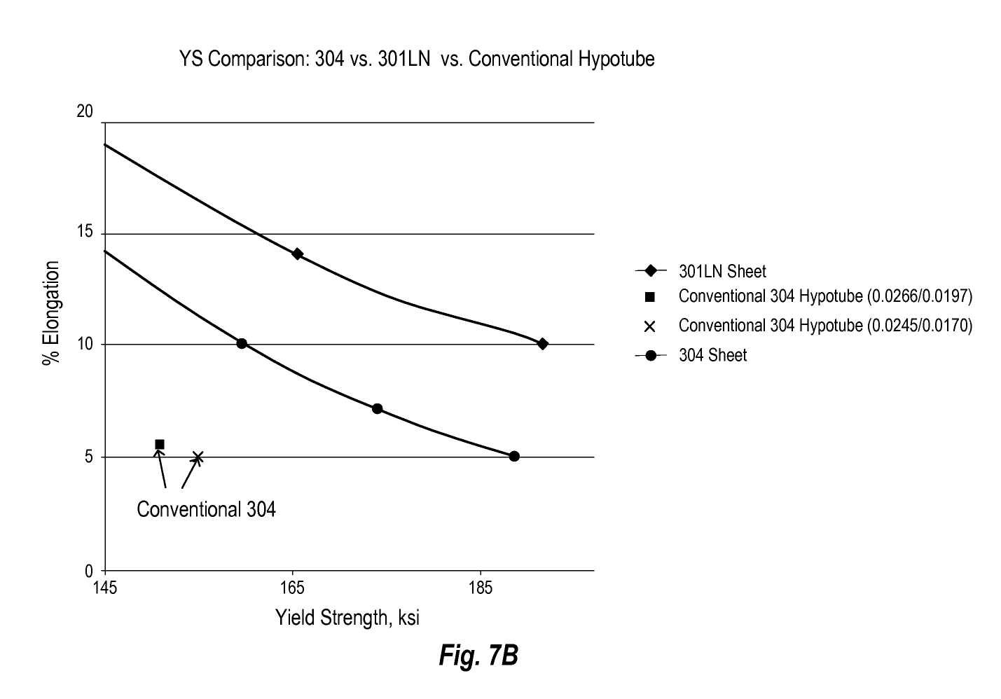

FIGS. 7A and 7B plot ductility and strength characteristics for AISI 304 stainless steel sheet (Uginox 18-9E, 18-9D, and 18-9DDQ), AISI 301LN stainless steel sheet (Uginox 18-7L), and also plot ductility and strength “point” values for several exemplary commercially available hypotubes, all of which are or described as being fabricated from AISI 304 stainless steel. For example, hypotubes fabricated from conventional AISI 304 stainless steel having OD/ID dimensions of 0.0266/0.0197 and 0.0245/0.0170 provide UTS values of 203 ksi and 201 ksi, respectively. The ductility of such hypotubes is 5.5% and 4.9%, respectively. The strength and ductility data for commercial AISI 304 stainless steel hypotubes plotted in FIGS. 7A-7B is also presented below, in Table 3.

TABLE 3

| | OD | | UTS | | | | Elongation |
|---|---|---|---|---|---|---|---|
| Sample | (in) | ID (in) | (MPa) | UTS (ksi) | YS (MPa) | YS (ksi) | (%) |
| Conventional 1 | 0.0266 | 0.0197 | 1400 | 203 | 1041 | 151 | 5.5 |
| Conventional 2 | 0.0245 | 0.0170 | 1386 | 201 | 1069 | 155 | 4.9 |

It is readily apparent that by forming the hypotube from a stainless steel alloy such as AISI 301LN, rather than AISI 304, that significantly better combinations of strength and ductility can be achieved, even without a static strain hardening treatment. Where static strain hardening is employed, the resulting combination of strength and ductility would be even better than that shown by the AISI 301LN curve in FIGS. 7A-7B. For example, the use of AISI 301LN may provide an additional 5-10 percentage points better ductility at a given UTS as compared to the commercial hypotubes formed of AISI 304 stainless steel.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A hypotube for use with an intra-corporal medical device, the hypotube comprising:
an elongate hollow body extending from a proximal end to a distal end;
at least a portion of the elongate hollow body being fabricated from a stainless steel alloy having: (1) a nitrogen content, a carbon content, or a combined nitrogen and carbon content that is greater than an upper specification limit for AISI 304 stainless steel and/or (2) an austenitic stabilizer content that is lower than a lower specification limit for AISI 304 stainless steel.

2. The hypotube of claim 1, wherein at least a portion of the elongate hollow body is fabricated from an AISI 300 series stainless steel alloy other than AISI 304 stainless steel.

3. The hypotube of claim 2, wherein at least a portion of the elongate hollow body is fabricated from an AISI 300 series stainless steel alloy selected from the group consisting of 304N stainless steel, 304LN stainless steel, 301 stainless steel, 301L stainless steel, 301LN stainless steel, 316N stainless steel, 316LN stainless steel, and 302 stainless steel.

4. The hypotube of claim 1, wherein at least a portion of the elongate hollow body is fabricated from a stainless steel alloy having a nitrogen content greater than 0.1% by weight.

5. The hypotube of claim 4, wherein at least a portion of the elongate hollow body is fabricated from a stainless steel alloy having a nitrogen content not greater than about 0.3% by weight.

6. The hypotube of claim 1, wherein at least a portion of the elongate hollow body is fabricated from a stainless steel alloy having a combined nitrogen and carbon content greater than 0.18% by weight.

7. The hypotube of claim 1, wherein at least a portion of the elongate hollow body is fabricated from a stainless steel alloy having a carbon content greater than 0.08% by weight.

8. The hypotube of claim 7, wherein at least a portion of the elongate hollow body is fabricated from a stainless steel alloy having a carbon content not greater than about 0.15% by weight.

9. The hypotube of claim 1, wherein at least a portion of the elongate hollow body is fabricated from a stainless steel alloy having a carbon content not greater than 0.03% by weight.

10. The hypotube of claim 1, wherein at least a portion of the elongate hollow body is fabricated from a stainless steel alloy having a nickel content that is lower than a lower specification limit of AISI 304 stainless steel.

11. The hypotube of claim 1, wherein at least a portion of the elongate hollow body is fabricated from a stainless steel alloy comprising molybdenum.

12. The hypotube of claim 1, wherein the stainless steel alloy exhibits higher ductility without any substantial decrease in yield strength as compared to AISI 304 stainless steel.

13. The hypotube of claim 1, wherein the stainless steel alloy has been strain aged at a temperature from about 150° F. to about 750° F. to increase yield strength as compared to the stainless steel alloy prior to strain aging.

14. A hypotube for use with an intra-corporal medical device, the hypotube comprising:
an elongate hollow body extending from a proximal end to a distal end;
at least a portion of the elongate hollow body being fabricated from a stainless steel alloy having a nickel content that is lower than a lower specification limit of AISI 304 stainless steel, and a nitrogen content that is greater than an upper specification limit of AISI 304 stainless steel.

15. The hypotube of claim 14, wherein at least a portion of the elongate hollow body is fabricated from a stainless steel alloy having no or negligible manganese and a nickel content that is not more than 8% by weight.

16. The hypotube of claim 14, wherein at least a portion of the elongate hollow body is fabricated from a stainless steel alloy having a nickel content from 6% to less than 8% by weight.

17. The hypotube of claim 14, wherein at least a portion of the elongate hollow body is fabricated from a stainless steel alloy having a nitrogen content greater than 0.10% by weight.

18. The hypotube of claim 14, wherein at least a portion of the elongate hollow body is fabricated from a stainless steel alloy having a carbon content not greater than 0.03% by weight.

19. The hypotube of claim 14, wherein at least a portion of the elongate hollow body is fabricated from a stainless steel alloy comprising molybdenum.

20. A hypotube for use with an intra-corporal medical device, the hypotube comprising:
an elongate hollow body extending from a proximal end to a distal end;
at least a portion of the elongate hollow body being fabricated from a stainless steel alloy having a nickel content that is from 6% to 8% by weight, and a nitrogen content that is greater than 0.10% by weight.
